(19) United States
(12) Patent Application Publication
(10) Pub. No.: US 2004/0234983 A1
(43) Pub. Date: Nov. 25, 2004

(54) Method for assessing the virulence of pathogens and uses thereof

(76) Inventors: Pierre Cosson, Carouge (CH); Jean-Pierre Paccaud, Sezenove (CH); Thilo Kohler, Carougw (CH)

Correspondence Address: MINTZ LEVIN COHN FERRIS GLOVSKY & POPEO, 666 THIRD AVENUE, NEW YORK, NY, 10017, US

(21) Appl. No.: 10/480,758

(22) PCT Filed: Jun. 7, 2002
(86) PCT No.: PCT/IB02/03277

(51) Int. Cl. C12Q 1/68; G01N 33/53

(52) U.S. Cl. 435/6

(57) ABSTRACT

The present invention relates to a method assessing the virulence of a pathogen or toxin to a host organism. The present invention further relates to a method to identify a composition that reduces the virulence of a pathogen or toxin to a host organism. The invention also relates to a method to identify genes encoding toxins or factors that are pathogenic to a host organism.

### FIELD OF THE INVENTION

The present invention relates to a method assessing the virulence of a pathogen or toxin to a host organism. The present invention further relates to a method to identify a composition that reduces the virulence of a pathogen or toxin to a host organism. The invention also relates to a method to identify genes encoding toxins or factors that are pathogenic to a host organism.

### BACKGROUND OF THE INVENTION

Pathogenesis involves the interaction of a pathogen with a host cell. It has been shown, that factors which affect pathogenesis in plants or animals can also affect pathogenesis in lower eukaryotic organisms. For example, the same bacterial virulence factors of Pseudomonas aeruginosa (P. aeruginosa), which affect the worm Caenorhabditis elegans (C. elegans), also affect Arabidopsis thaliana and show virulence in a mammalian mouse assay (WO 98/50080). Also, a method for identifying common virulence genes using C. elegans as a host organism has been described for identifying compounds that repress virulence of a pathogenic agent (WO 98/50080).

However, approaches for testing virulence using multicellular organisms are time-consuming and expensive. In addition, the use of organisms such as C. elegans, are disadvantaged by the fact that there is never any direct contact between the pathogen and the host cells because the worm is surrounded by a cuticle, and its digestive tract is lined with a peritrophic membrane. These barriers limit the use of C. elegans virulence testing to the assessment of diffusible factors that can contact the worm and potentially interfere with its physiology. This is a serious limitation because many pathogens (e.g., bacterial pathogens) act intracellularly. The use of C. elegans in virulence testing is also limited by the high costs of culture and the time required to grow and prepare the organism for the screening test. Furthermore, interpretation of test results with organisms such as C. elegans, can be difficult due to the complexity of the organisms. The assessment of pathogenicity in mammalian hosts is even more time-consuming and expensive.

Therefore, there is a need in the art for more time-efficient and cost-efficient alternative test host systems for testing pathogen virulence.

### SUMMARY OF THE INVENTION

The present invention provides a method to assess the virulence of a pathogen or a toxin to a unicellular host organism. The use of a unicellular test host organism (such as Dictyostelium) as a host model offers several advantages. First, the simplicity and reproducibility of the unicellular test host organism system surpass that of mammalian systems, as well as other non-mammalian systems. Secondly, a unicellular test host system represents a powerful genetic system to analyze host-pathogen relationships. Indeed, efficient genetic tools are available, allowing the isolation of unicellular test host mutants with increased or decreased sensitivity to pathogens, and the identification of the corresponding genes. Thus, the present invention provides a more time and cost efficient model for assessing virulence of pathogens and/or toxins.

According to the present invention, the virulence of a pathogen or a toxin to a host organism is assessed by exposing a unicellular test host organism to a pathogen or a toxin and monitoring the growth of test host organism. The virulence of a pathogen or a toxin is proportional to the level of efficiency of inhibition of the unicellular test host organism growth (i.e., the more efficient a pathogen or toxin is at inhibiting the growth of the unicellular test host organism, the higher the virulence of the pathogen or toxin). In one embodiment, a constant amount of the test host organism cell is incubated with various concentrations of a pathogen or toxin (e.g., serial dilution). In an alternate embodiment, various amounts of the test host organism (e.g., serial dilution) are assessed with a constant concentration of pathogen or toxin.

In another aspect, the present invention further provides a method for comparing the virulence to a unicellular test host organism of two pathogens. The method comprises exposing separate cultures of a unicellular test host organism to the two pathogens and monitoring the growth of each culture. The level of growth inhibition induced by the two pathogens are compared, and the pathogen exhibiting the higher level of inhibition has the higher virulence to the unicellular test host organism.

In yet another aspect, the present invention provides a method to identify a composition that reduces the virulence of a pathogen or toxin to a test host organism. The method comprises exposing a unicellular test host organism to a pathogen or toxin independently in the presence and in the absence of a candidate composition and then monitoring the growth of the unicellular test host organist A higher level of growth of the unicellular test host organism in the presence of the candidate composition than in the absence of the candidate composition indicates that the candidate composition reduces the virulence of the pathogen to the unicellular test host organism.

In yet another aspect of the present invention, genes encoding factors that are pathogens or toxins to a host organism may be identified by comparing the growth of a unicellular test host organism in the presence and absence of a test organism, or the product of a test organism, with a known or identifiable genetic alteration.

The methods of the present invention are useful in identifying and assessing any virulence factor affecting the growth or health of a unicellular test host organism. In one embodiment, the test host organism is exposed to two or more concentrations of the pathogen or toxin. In an alternative embodiment, two or more concentrations of the test host organism are exposed a single concentration of the pathogen or toxin.

For the methods of the present invention, the preferred unicellular test host organism is an amoebae species, such as, but not limited to, DICTYOSTELIUM SPECIES, Entamoeba species or Acanthamoeba species. A preferred amoeba is Dictyostelium, preferably D. discoideum, more preferably D. discoideum DH1. The pathogen can be bacteria, mycobacteria, fungi, and unicellular eukaryotic organism. The pathogen can be an extracellular pathogen or an intracellular pathogen (e.g., a bacteria which is not killed efficiently). Bacterial pathogen include, for example, Pneumococci sp., Klebsiella, sp., Pseudomonas, e.g., P. aeruginosa, Salmonella, e.g., Salmonella typhimurium, Legionella, e.g., Legionella pneumophilia, Escherichia, e.g., Escherichia coli, Listeria, e.g., Listeria monocytogenes, Staphylococcus, e.g., Staphylococcus aureus, Streptococci sp., Vibrio, e.g., Vibrio cholerae. The virulence of the pathogen can be triggered by a quorum-sensing pathway. Pathogenic mycobacteria include e.g., Mycobacterium tuberculosis. Pathogenic fungi include, e.g., Candida albicans. Pathogenic unicellular eukaryotic organisms include, e.g., Leishmania donovani.

These and other objects of the present invention will be apparent from the detailed description of the invention provided below.

### BRIEF DESCRIPTION OF THE DRAWINGS

The present invention will be further understood from the following description with reference to the figures which:

FIG. 1 demonstrates the inhibition of D. discoideum growth by P. aeruginosa and the role of quorum-sensing systems. Approximately 200 D. discoideum cells were plated with a lawn of Klebsiella pneumoniae (K. pneumoniae) bacteria (A), supplemented with P. aeruginosa strains PT5 (WT, B), PT462 (rhlR, C), PT498 (lasR, D), PT531 (rhlR, lasR, E) or PT712 (rhlA, F). Growth of D. discoideum colonies was observed after 5 days.

FIG. 2 depicts the quantitative assessment of D. discoideum growth on P. aeruginosa. Decreasing numbers of D. discoideum cells from 50.000 (upper left) to 1 (middle, down) were plated on a lawn of P. aeruginosa and allowed to grow for 5 days. A, PT5; B, PT498 (lasR); C, PT462 (rhlR); D, PT531 (lasR, rhlR).

FIG. 3 demonstrates the effect of P. aeruginosa supernatants on the actin cytoskeleton of Dictyostelium cells. Dictyostelium cells were exposed for 1 hr to HL5 (A) or to the supernatant of early stationary P. aeruginosa PT5 bacteria (B) or PT531 bacteria (C). Alternatively the cells were exposed to PT5 bacteria in fresh HL5 medium (D). The cells were then fixed and their actin stained with Texas Red-phalloïdin.

FIG. 4 demonstrates the lysis of Dictyostelium cells exposed to concentrated Pseudomonas supernatants. Dictyostelium cells were exposed to culture supernatants of wild type strain PT5 (A, upper panels) and the double mutant PT531 (A, lower panels) and observed in a phase contrast microscope. Arrows indicate individual Dictyostelium cells being lysed by wild type supernatant during the indicated time frame. The kinetics of cell lysis induced by supernatants of the wild type (PT5), the lasR-rhlR double mutant (PT531) and the rhlA mutant (PT712) was determined by counting Dictyostelium cells under the microscope in a field containing approximately 100 cells at t=0 (B).

### DETAILED DESCRIPTION OF THE INVENTION

Definitions

As used herein, each of the following terms has the meaning associated with it in this section.

The term “pathogen,” as used herein, is intended to include an agent that causes disease, especially a living microorganism such as a bacterium or fungus. The terms “agent” and “factor” are used interchangeably herein to describe pathogens or toxins useful in the methods of the present invention. Pathogens may include any bacteria, mycobacteria, fungi and unicellular eukaryotic organism, including wild types and mutants thereof, which causes disease or brings about damage or harm to a host organism Pathogens may also be a poisonous substance, e.g., toxin, that is produced by living cells or organisms and is capable of causing disease when introduced to a host.

The term, “pathogenicity,” as used herein, is defined as an agent's ability to cause disease, damage or harm to a host organism.

The term, “virulence,” as used herein, is a measure of the degree of pathogenicity of an agent to a host organism. Virulence is usually expressed as the dose of an agent or cell number of a pathogen that will elicit a pathological response in the host organism within a given time period. “Reducing the virulence” as used herein is defined as the ability of a compound to attenuate, diminish, decrease, suppress, or arrest the development of, or the progression of disease, damage or harm to a host organism mediated by a pathogen.

The term, “host organism,” as used herein, is intended to include any living organism. Preferably the host organism is a eukaryote, e.g., vertebrate. More preferably the host organism is a mammal. It is, most preferred that the host organism be a human.

The term, “unicellular test host organism,” as used herein, is intended to include any living unicellular organism, including, but not limited to, amoebae, flagellates, ciliates, and other protozoal parasites. Preferably the organism is an amoeba, such as, but not limited to, DICTYOSTELIUM SPECIES, Entamoeba species, or Acanthamoeba species. A preferred amoeba is Dictyostelium, preferably D. discoideum, more preferably D. discoideum DH1.

I. Pathogens of the Present Invention

Bacterial pathogens of the present invention may include Pneumococci sp., Klebsiella, sp., Pseudomonas, e.g., P. aeruginosa, Salmonella, e.g., Salmonella typhimurium, Legionella, e.g., Legionella pneumophilia, Escherichia, e.g., Escherichia coli, Listeria, e.g., Listeria monocytogenes, Staphylococcus, e.g., Staphylococcus aureus, Streptococci sp., Vibrio, e.g., Vibrio cholerae. Pathogenic mycobacteria of the present invention may include e.g., Mycobacterium tuberculosis. Pathogenic fungi of the present invention may include, e.g., Candida albicans. Pathogenic unicellular eukaryotic organisms of the present invention may include, e.g., Leishmania donovani.

The present invention provides a method useful in identifying and assessing any virulence factor affecting the growth or health of a host organism. The virulence factor can be an intracellular pathogen of the unicellular test host organism, e.g., D. discoideum, such as an intracellularly growing bacterial pathogen e.g., select strains of Salmonella, Legionella, or Listeria, or mycobacterial pathogen, e.g., Mycobacterium tuberculosis. Alternatively, the pathogen may be an extracellular pathogen that grows outside of the unicellular test host organism, such as select strains of Pseudomonas, e.g., P. aeruginosa, Escherichia, e.g., Escherichia coli, Staphylococcus, e.g., Staphylococcus aureus, Vibrio, e.g., Vibrio cholerae, or an extracellularly growing pathogenic fungi, e.g., Candida albicans.

Usually the pathogen is a bacterial pathogen. Preferably, the pathogen is an extracellular bacterial pathogen. More preferably, the pathogen is Pseudomonas. Most preferably the pathogen is P. aeruginosa.

The virulence of the pathogen might be triggered by the quorum sensing pathway which is used by a broad range of pathogenic bacteria e.g., P. aeruginosa, Escherichia coli, Staphylococcus aureus, Vibrio cholerae, Enterococcus sp., and Mycobacterium sp., whereby a threshold concentration of autoinducer triggers the coordinated synthesis of secreted virulence factors which initiates the pathogenic process. Preferably, the virulence of the pathogen is triggered by the quorum sensing pathway.

II. Methods of Exposing a Unicellular Test Organism to a Pathogen

In one embodiment, the unicellular test host organism is exposed to a pathogen by mixing cells from a culture of the unicellular test host organism with cells from a culture of the pathogen. This mixture is then promptly plated on agar plates. In another embodiment, serial dilutions of the unicellular test host organism cells are applied to an agar plate [e.g., SM-agar (Standard Medium, Kenneth B. Raper, 1984, The Dictyostelids, Princeton University Press, p.59] with a pregrown lawn of pathogenic cells. In yet another embodiment, a pathogen is added to the unicellular test host organism cells cultured on a surface (e.g., a glass coverslip). Adding dilutions of cells from a culture of the unicellular test host organism to a lawn of pathogen cells is, however, a preferred method of virulence testing. Any appropriate method and conditions of culture of a unicellular test host organism known to the artisan may be used in method of the present invention, e.g., liquid culture of Dictyostelium.

Test sample preparations may include the supernatant of a pathogen cell culture. This is particularly useful if the virulence factors are secreted into the culture media. For example, conditions that triggered the quorum sensing pathway can result in the secretion of virulence factors from pathogenic bacteria.

Under certain culture conditions, the unicellular test host organism may use a pathogen as a growth substrate. The ability of the unicellular test host organism to grow on a pathogen substrate depends on the character of the pathogen, e.g., pathogen virulence. Other bacterium, e.g., K. pneumoniae, may be added to a culture of the test host organism exposed to the pathogen and serve as a growth supplement for the amoebas. The growth supplement may be either a pathogenic or non-pathogenic bacteria.

Generally unstarved unicellular test host organism cells are used in the methods of the present invention. For example, a test host organism such as D. discoideum can be cultured in adherent culture or in suspension culture for use in the methods of the present invention. HL5 medium (Cornillon et al., 1994, J. Cell Sci. 107, 2691-2704) is useful to grow the D. discoideum cells for use in the present invention. Any culture medium that will sustain the growth of D. discoideum may be used to produce the amoeba cells. Also, the strain of D. discoideum is not critical to the methods of the present invention. Dictyostelium strains such as DH1 (Cornillon, et al., 2000. J. Biol. Chem. 275, 34287-92), AX-2, or AX-3 (both referenced in Kenneth B. Raper, 1984, The Dictyostelids, Princeton University Press, 7475) can be used in the present invention. The DH1 Dictyostelium is a preferred strain for use in the methods of the present invention.

The conditions of pathogen culture are not critical to the methods of the present invention. Such conditions may be tailored to the pathogen being tested and are described in the art, e.g., for bacteria in Bergey's Manual of Systematic Bacteriology (The Prokaryotes 2^{nd} edition by A. Balows et at., 1992). Under conditions where the virulence of the pathogen is triggered by the quorum sensing pathway, the pathogen may be grown to a cell density at which the induction of the quorum sensing is maximal.

III. Methods of Monitoring the Growth of the Unicellular Test Host Organism

The unicellular test host organism cell growth, e.g., Dictyostelium cell growth, may be monitored in many ways known to one skilled in the art. How cell growth is monitored depends, in part, on the method of pathogen exposure used. If the mixture of unicellular test host organism and pathogen (or a combination of pathogens) are cultured on agar plates, the agar plate may be incubated under conditions suited to colony growth and measurement (e.g., colony size and number). For example, D. discoideum and pathogen (or a combination of pathogens) may be cultured for 4 to 7 days (at 19° C. to 25° C.) in the presence test compound. After the incubation period the occurrence, number, and the size of colonies formed in the presence of test compound are recorded and compared with the occurrence, number, and size of colonies formed in the absence of the test compound. The size of the colonies may be measured by any means suitable to determine colony dimension. Determining colony size as by measuring the growth zone as reflected by the colony diameter is useful in the methods of the present invention.

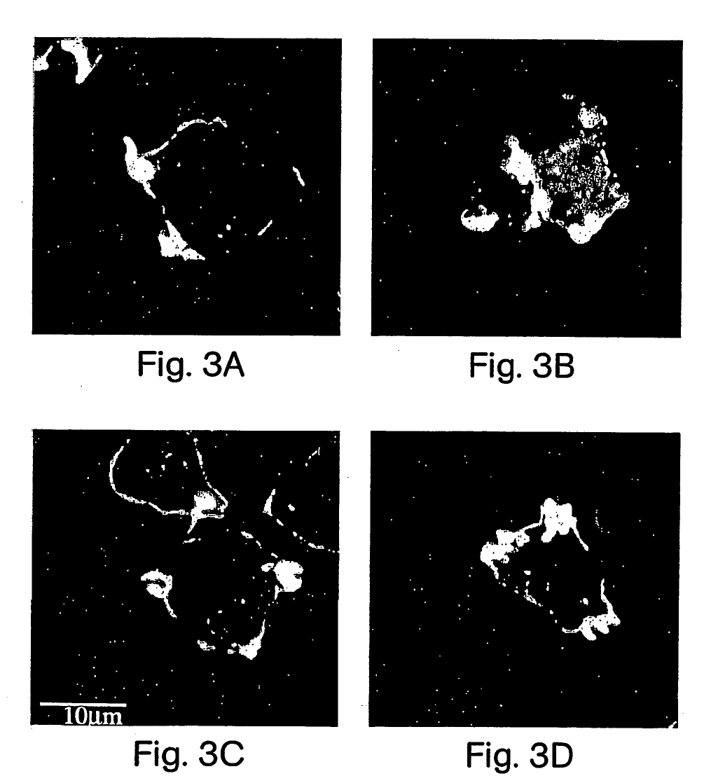

Unicellular test host organism growth can also be monitored by actin staining used to visualize the unicellular test host organisms' cytoskeleton with a light microscope (e.g., see infra FIG. 3). This monitoring approach is particularly useful where the unicellular test host organism is cultured on a surface (e.g., a glass coverslip) prior to the pathogen addition. Defects in the organization of the unicellular test host organism's actin cytoskeleton may be used to monitor disruption of cell growth. A loss of cortical actin staining or the presence of patches of polymerised actin can be taken as an indices of growth. Alternatively, growth of the unicellular test host organism may be monitored fluorimetrically by well-known analytical techniques quantifying the total fluorescence emitted by a culture of unicellular organisms. It is common knowledge in the art that the level of fluorescence in culture test samples is directly proportional to the rate of cell growth, e.g., Dictyostelium. Alternatively, any method known in the art to monitor the growth of a unicellular test host organism may be used in the method of the present invention, e.g., cell counting or measurement of reporter gene expression.

IV. Character and Potency of Anti-Virulence Compounds of the Present Invention

A. Character of Anti-Virulence Compounds of the Present Invention

A compound which reduces the virulence of a pathogen to a host organism can include any synthetic or semi-synthetic compound. Such compounds include inorganic as well as organic chemical compounds. The compounds may be naturally occurring. Naturally occurring compounds may include, e.g., saccharides, lipids, peptides, proteins, nucleic acids, or combinations thereof, e.g., aminoglycosides, glycolipids, lipopolysaccharides, or macrolides. The precise source of the compound is not critical to the method of the present invention. The compound might be derived from e.g., synthetic compounds libraries which are commercially available, e.g., Sigma-Aldrich (Milwaukee, Wis.), or libraries of natural occurring compounds in the form of bacterial, fungal, plant, and animal extracts such as those available from Xenova (Slough, UK). The synthetic (or semi-synthetic) or natural occurring compounds might be modified using standard chemical, physical, or biochemical methods known in the art.

The order of test compound addition is not critical to the methods of the present invention. A test compound may be added to the pathogen before unicellular test host organism cells, are added to the assay mixture. While this is the preferred order of addition, test compound may also be added to the assay mixture after the pathogen contacts the unicellular test host organism cells.

Test compound may be produced in the course of assay by a pathogen present in the unicellular test host assay mixture. Alternatively, unicellular test host organism cells may be preincubated with test-compound-producing pathogen cells prior to exposure to a pathogen that does not produce test compound.

In the methods of the present invention, one or more test compounds may be present or produced in the assay mixture. Preferably one compound is present, or produced, in the assay mixture.

B. Potency of Anti-Virulence Compounds

A compound with anti-virulence activity increases the growth of the unicellular test host organism cells exposed to a pathogen. That is, unicellular test host organism cell growth (e.g., D. discoideum growth), in the presence of a pathogen, is improved by contact with a test compound, or mixture of test compounds, when compared with the growth of unicellular test host organism cell growth not contacted test compound. Depending on the conditions of assay, an anti-virulent compound may display a range of growth promoting activity in the methods of the present invention. Growth of unicellular test host organism challenged with a pathogen may be 3-fold or greater in the presence of an anti-virulence active compound than amoebal cell growth observed in control assay mixtures without the anti-virulence compound. Rat mortality assays such as that described by Join-Lambert et al. (2001, Antimicrob. Agents Chemother., 45(2):571-6) can be used to corroborate anti-virulence activity observed in the assay methods of the present invention.

V. Use of the Methods of the Present Invention to Identify Genes Encoding Virulence Factors

In another embodiment of the present invention, genes encoding virulence factors (e.g., pathogens or toxins) to a host organism may be identified by comparing the growth of a unicellular test host organism, e.g., D. discoideum, in the presence and absence of a test organism, or the product of a test organism, with a known or identifiable genetic alteration. To identify such genes, a pathogen is selected which inhibits the growth of the unicellular test host organism. Mutants of the pathogen are produced by standard techniques well-known in the art. The unicellular test host organism is contacted with the mutant pathogen and assessed for growth. Mutations yielding reduced virulence are identified where the growth of the unicellular test host organism exposed to the mutant pathogen is greater than the growth of unicellular test host organism exposed to wild-type pathogen. Specific genetic mutations in pathogens displaying reduced virulence are identified and characterized by techniques well-know in the art. Thus, genes encoding virulence factors (e.g., pathogens or toxins) to a host organism may be identified using the methods of the present invention.

The pathogen which inhibits the growth of the unicellular test host organism can be selected by exposing unicellular test host organism to different pathogens and monitoring the growth of the unicellular test host organism, as indicated above. Any pathogen affecting the unicellular test host organism growth may be mutated. Usually, the mutated pathogen is a bacterial pathogen. Preferably, the mutated pathogen is an extracellular bacterial pathogen. More preferably, the mutated pathogen is a Pseudomonas pathogen. The virulence of the pathogen may be triggered by the quorum sensing system described above.

The mutant can be generated according to known methods in the art such as ultraviolet radiation exposure, treatment with intercalating agent or transducing phage or a transposon insertion. On the other hand, mutants already known in the art can be used. The mutation can be used as a marker employing methods known in the art to further identify the virulence factor.

### DETAILED DESCRIPTION OF THE DRAWINGS

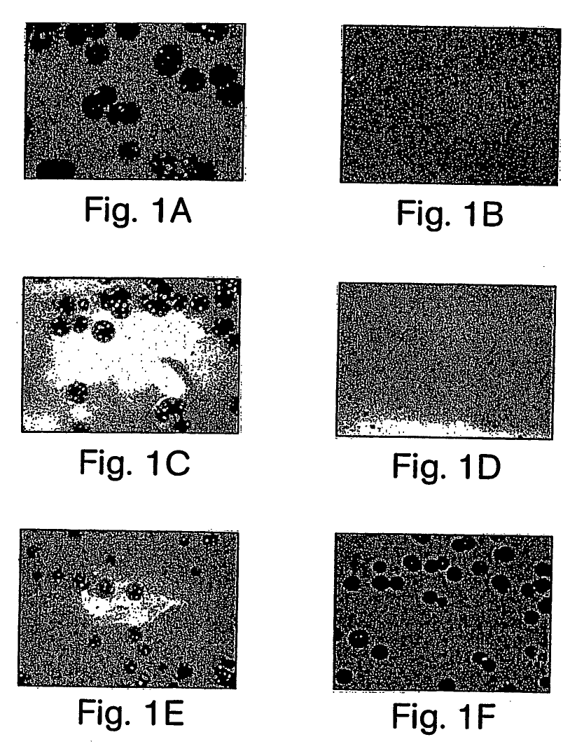

FIG. 1 demonstrates the inhibition of i D. discoideum growth by P. aeruginosa and the role of quorum-sensing systems. Approximately 200 D. discoideum cells were plated with a lawn of Klebsiella pneumoniae (K. pneumoniae) bacteria (A), supplemented with P. aeruginosa strains PT5 (WT, B), PT462 (rhlR, C), PT498 (lasR, D), PT531 (rhlR, lasR, E) or PT712 (rhlA, P). Growth of D. discoideum colonies was observed after 5 days.

The inhibition of D. discoideum growth by P. aeruginosa was assayed by mixing 200 D. discoideum cells with 300 μl (6×10⁸ cfu) K. pneumoniae and 10 μl (10⁷ cfu) P. aeruginosa culture and plating immediately on SM-agar (Cornillon et al., 1994 J. Cell Sci., 107 (Pt 10), 2691-704). The plates were then incubated for five days at 25° C. Alternatively D. discoideum were plated with 200 μl (2×10⁸ cfu) of P. aeruginosa alone.

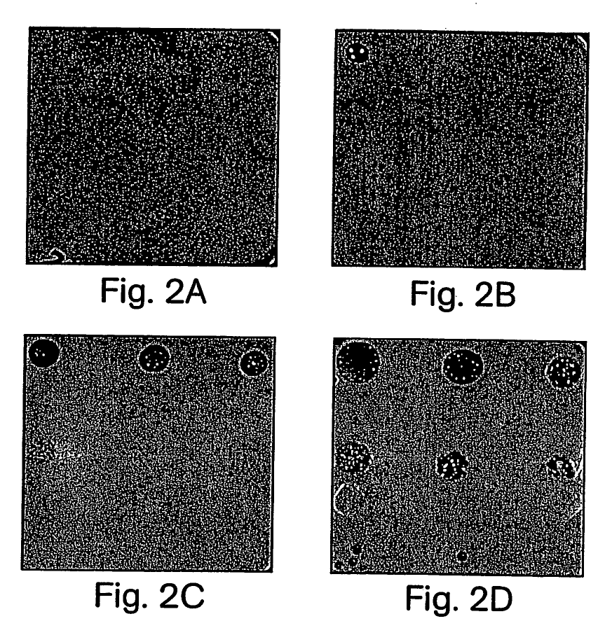

FIG. 2 details the quantitative assessment of D. discoideum growth on P. aeruginosa. Decreasing numbers of D. discoideum cells from 50.000 (upper left) to I (middle, down) were plated on a lawn of P. aeruginosa and allowed to grow for 5 days. A, PT5; B, PT498 (lasR); C, PT462 (rhlR); D, PT531 (lasR, rhlR).

Quantitative measurements of D. discoideum growth on a lawn of pure P. aeruginosa. were obtained by first plating 200 μl of P. aeruginosa culture on SM-agar. Droplets of 5 μl containing serial dilutions of D. discoideum cells (50.000 cells, 10.000 cells, 2.000, etc.) were then spotted on the bacterial lawn. The plates were incubated for 5 days at 25° C. and the highest dilution at which D. discoideum growth was visible was recorded.

FIG. 3 demonstrates the effect of P. aeruginosa supernatants on the actin cytoskeleton of Dictyostelium cells. Dictyostelium cells were exposed for 1 hr to HL5 (A) or to the supernatant of early stationary P. aeruginosa PT5 bacteria (B) or PT531 bacteria (C). Alternatively the cells were exposed to PT5 bacteria in fresh HL5 medium (D). The cells were then fixed and their actin stained with Texas Red-phalloïdin.

To visualize the effect of bacterial supernatants on the actin cytoskeleton, Dictyostelium cells were grown on glass coverslips for three days, then exposed either to bacterial supernatants (6 hr growth) or to bacteria in fresh HL5 (10 bacteria per cell) for 1 hour. The cells were then fixed with paraformaldehyde (4%; 30 min), permeabilized in saponin, and the actin cytoskeleton was visualized with Texas Red-labeled phalloïdin (Molecular Probes). The cells were imaged with a Zeiss LSM 510 laser scanning confocal microscope.

When supernatants of overnight bacterial cultures were used, D. discoideum cells were observed in phase contrast with a Zeiss Axiovert 100 microscope, and pictures recorded every 30 sec with a Hamamatsu Orca camera and analyzed with OpenLab 3 software. Cells were counted and their number plotted as a function of the time after addition of bacterial supernatant.

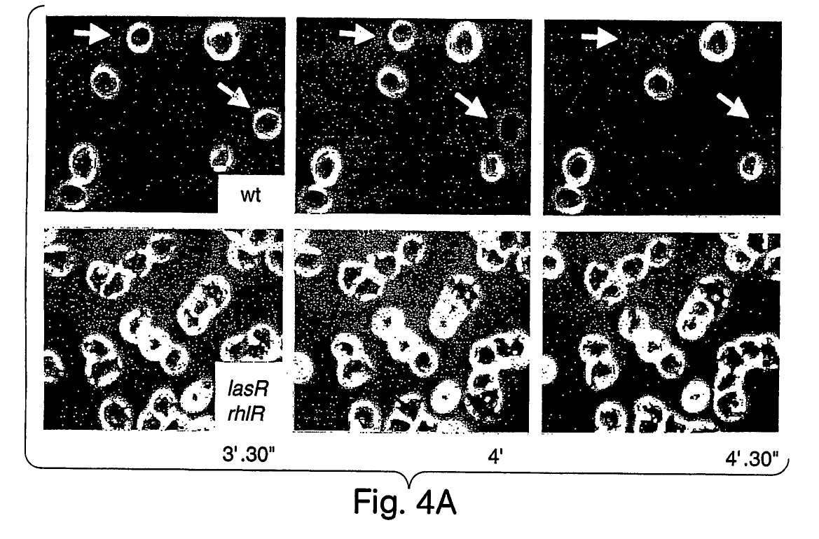

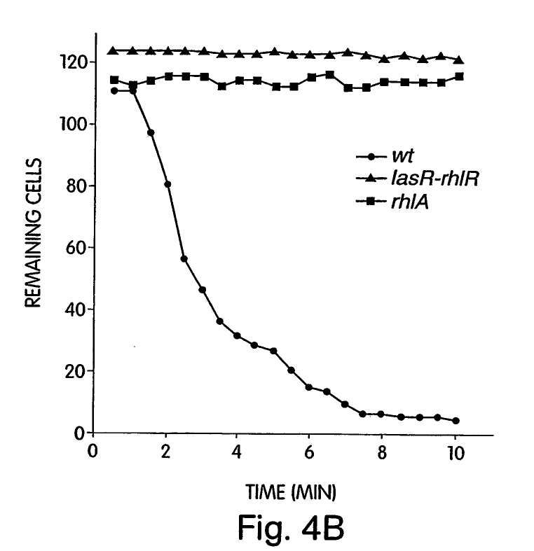

FIG. 4 demonstrates the lysis of Dictyostelium cells exposed to concentrated Pseudomonas supernatants. Dictyostelium cells were exposed to culture supernatants of wild type strain PT5 (A, upper panels) and the double mutant PT531 (A, lower panels) and observed in a phase contrast microscope. Arrows indicate individual Dictyostelium cells being lysed by wild type supernatant during the indicated time frame. The kinetics of cell lysis induced by supernatants of the wild type (PT5), the lasR-rhlR double mutant (PIT531) and the rhlA mutant (PT712) was determined by counting Dictyostelium cells under the microscope in a field containing approximately 100 cells at t=0 (B). Pseudomonas bacteria (PAO1, PT531 or P712) were grown in HL5 medium at 37° C. for either 6 hours or overnight. The bacteria were then pelleted by centrifugation (10 min at 7,000×g) and the supernatant collected and filtered (0.22 μm).

### EXAMPLES

These Examples are provided for the purpose of illustration only and the invention should in no way be construed as being limited to these Examples, but rather should be construed to encompass any and all variations which become evident as a result of the teaching provided.

### Example 1

### Strains and Culture Conditions Used in the Present Studies

The D. discoideum wild-type strain DH1-10 used in this study is a subclone of DH1 (Cornillon et al., 2000, J. Biol. Chem., 275(44), 34287-92). Cells were grown at 21° C. in 1U5 medium (14.3 g/l peptone (Oxoid), 7.15 g/l yeast extract, 18 g/l maltose, 0.64 g/l Na₂HPO₄⁻2H₂O, 0.49 g/l KH₂PO₄, pH 6.7) (Cornillon et al., 1994, J. Cell. Sci., 107 (Pt 10), 2691-704) an subcultured twice a week.

Bacterial strains used in this study are described in Table 1. The genotypes of the three efflux pump overexpressing mutants, PT149 (NfxC, MexEF-OprN overexpressor), PT625 (NalC, MexAB-OprM overexpressor) and PT648 (nfxB, MexCD-OprJ overexpressor), were determined by sequencing the corresponding regulator gene. The strains PT149 (NfxC) and PT637 (NfxC, mexE) were described recently in detail (Join-Lambert et al., Antimicrob. Agents Chemother., 45, 571-6. Briefly, the mexT transcriptional activator gene (Maseda et al., FEMS Microbiol. Lett., 192, 107-12) is interrupted by an 8 bp insertion in our P. aeruginosa wild-type strain PT5. In the NfxC mutant PT149, the 8 bp insert is not present yielding a functional mexT gene able to cause overexpression of the MexEF-OprN efflux system. To obtain PT637, the mexE gene was inactivated in PT149, restoring wild-type antibiotic susceptibility. PT625 did not contain any mutation in the mexR regulator gene of the mexAB-oprM efflux operon. However since the strain was shown by Western blot analysis to overexpress the MexAB-OprM efflux pump (Köhler et al., unpublished observation) the strain was considered to be a NalC mutant (40). Strain PT648, which overexpresses the MexCD-OprJ system, was found to contain a two bp (AC) deletion at codon 19 of its cognate repressor gene nfxB, resulting in overexpression of MexCD-OprJ (nfxB phenotype).

Bacteria were grown overnight at 37° C. on Luria-Bertani (LB) agar. Single colonies were inoculated into 5 ml PB (2% (wt/vol) peptone, 0.3% (wt/vol) MgCl₂.6H₂O, 1% (wt/vol) K₂SO₄) (Essar et al., 1990, J. Bacteriol., 172(2),884-900) in a 50 ml flask and grown at 37° C. for 8 hr prior to use. The growth of various strains was tested in rich medium (PB) by measuring the optical density (600 nm) of a culture at different times after inoculation and was found to be comparable for all strains used. Under these conditions, similar OD₆₀₀ₛ were obtained for each strain and the induction of quorum sensing was maximal. Minimal Inhibitory Concentrations (MICs) were determined in Mueller-Hinton broth by the microdilution method (Thornsberry et al., 1983 NCCLS, 3, 48-56).

### Example 2

### P. Aeruginosa Inhibits Dictyostelium Growth: Role of the RHL Quorum-Sensing System

Dictyostelium amoebae are unicellular organisms that feed phagocytically upon bacteria such as K. pneunoniae. When 200 Dictostelium cells are plated with K. pneumoniae bacteria, each amoeba creates a plaque in the bacterial lawn, where bacteria have been phagocytosed (FIG. 1A). In contrast, addition of wild-type P. aeruginosa strain PT5 to the lawn of K. pneumoniae bacteria completely inhibited the growth of the amoebae (FIG. 1B). These results are in agreement with earlier studies which demonstrated that P. aeruginosa was a particularly inadequate bacterial growth substrate for D. discoideum (Raper et al., 1939, J. Bacteriol., 38, 16-32, 1632; Depraitère et al., 1978, Ann. Microbiol. (Paris), 129 B(3):451-61).

The relationship of this growth inhibition to the production of known virulence factors controlled by the quorum-sensing system was investigated. Interestingly, P. aeruginosa mutants affected in the rhl system, namely the rhlR mutant PT462 caused no inhibition of D. discoideum growth in our plating test as shown in FIG. 2C (Table 2). The las system, however, was not as important for the inhibition of Dictyostelium growth, since the lasR mutant PT498 allowed only very reduced growth of Dictyostelium (FIG. 1D).

However, in these experiments, Dictyostelium was grown in the presence of both Klebsiella and Pseudomonas bacteria. Thus, it was not clear whether the observed inhibition of Dictyostelium growth in the presence of Pseudomonas was a direct pathogenic effect on the amoeba or a secondary effect caused by Pseudomonas killing or inhibiting the growth of the nutrient lawn of K. pneumoniae. To rule out the latter possibility, the ability of D. discoideum cells to grow on a lawn of P. aeruginosa only was tested (Table 2). Individual colonies of amoebae grew only when particularly permissive strains of P. aeruginosa were used such as the rhlR, lasR double mutant PT531. To obtain more quantitative results, higher numbers of Dictyostelium cells were used to determine how many amoebae would be necessary to create a plaque in a lawn of P. aeruginosa. For this, eight droplets were applied on a lawn of pure P. aeruginosa, each droplet containing a defined number of Dictyostelium cells. Even 50.000 Dictyostelium cells failed to create a plaque in a lawn of wild-type P. aeruginosa (FIG. 2A). In a scale measuring the growth of Dictyostelium, this was noted as a zero. When the lasR mutant PT498 was used, 50.000 Dictyostelium cells created a plaque while the next dilution (10.000 cells) failed to do so (FIG. 2B). Consequently growth of Dictyostelium on the lasR mutant was scored as 1. On a lawn of the rhlR mutant PT462, amoebal growth could be observed in the first five dilutions of Dictyostelium (FIG. 2C; scored 5). The double mutant rhlR-lasR was fully permissive for Dictyostelium growth and amoebae growth could be observed even in the most diluted conditions (1 cell per drop in drop 8; FIG. 2D). These results define a scale from 0 to 8, where the most permissive strains of P. aeruginosa are scored 8 (FIG. 2D) and the least permissive 0 (FIG. 2A).

Using this test it was apparent that the rhl quorum-sensing system was essential for bacterial pathogenicity, since both rhlR and rhlI mutants were much more permissive than wild-type cells for Dictyostelium. Mutations in lasR and lasI only resulted in a minor decrease in pathogenicity, although it should be noted that when combined with a rid mutation they resulted in fully permissive strains (Table 2).

### Example 3

### Effect of Secreted Virulence Factors in D. discoideum Cells

A number of virulence factors placed under the control of the quorum sensing systems are secreted by Pseudomonas bacteria, for example pigments (pyocyanin), rhamnolipids or proteases. To test the hypothesis that secreted factors are responsible for the growth inhibition of Dictyostelium, filtered culture supernatants of the wild type strain PAO1 and the lasR-rhlR double mutant PT531 were prepared and their effect on Dictyostelium was tested. Following an 1 hour incubation of Dictyostelium cells with the supernatant of wild type strain PT5 (early stationary phase, i.e., 6 hours growth) severe defects in the organization of the actin cytoskeleton were observed (FIG. 3B) with a loss of the typical cortical actin staining. Incubation with the supernatant of PT531 bacteria had no effect on the actin cytoskeleton (FIG. 3C). No effect was seen when Dictyostelium cells were incubated with PT5 resuspended in fresh HL5 medium (FIG. 3D).

To test whether secreted factors are at least in part responsible for the growth inhibition of Dictyostelium, filtered culture supernatants of the wild type strain and of the lasR-rhlR double mutant PT531 were incubated with Dictyostelium cells. Examination by phase contrast microscopy showed a rapid lysis of Dictyostelium cells completed after a 10 min exposure to wild type supernatants (FIG. 4A, upper panels). Under the same conditions, the supernatant of the lasR-rhlR double mutant PT531 did not induce significant lysis of Dictyostelium cells (FIG. 4A, lower panels). These results indicate that wild-type bacteria secrete, under the control of the quorum-sensing systems, one or several factors that disrupt the Dictyostelium cells and lead to fast lysis.

Since mutants in the rhl system were particularly permissive for Dictyostelium growth, rhamnolipids, whose synthesis depends mainly on the rhl system, were tested to see whether they were involved in the fast lysis of Dictyostelium cells. Therefore, the effect of supernatants from the rhlA mutant PT712, which is specifically defective in rhamnolipid synthesis but is not affected in the quorum sensing circuit (Kohler et al., J. Bacteriol., 182, 5990-6) were tested. Rhamnolipids are rhamnose-containing glycolipids that act both as biosurfactants and hemolytic factors (Johnson et al., 1980, Infect, Immun., 29(3), 1028-33) and are produced under the control of the rhl quorum sensing system.

Supernatant from this rhlA mutant did not cause lysis of Dictyostelium cells (FIG. 4B), suggesting that rhamnolipids are essential for this effect Rhamnolipids were then purified from the supernatants of the wild type strain PT5 and showed that these rhamnolipids at a final concentration of 10 μg/ml were able to cause lysis of the Dictostelium cells (data not shown).

Together, these results demonstrate that rhamnolipids play a role in inhibiting Dictyostelium growth by inducing lysis of these cells. However, since the rhlA mutant was only partially permissive for Dictyostelium growth (Table 2), it is possible that several other exoproducts whose genes are under the control of the quorum-sensing systems also participate in the inhibition of Dictyostelium growth.

Example 4

### NfxC Mutants Reduced Virulence

In previous studies, the rhl quorum-sensing system has usually been considered less important for Pseudomonas virulence than the las quorum-sensing system. To compare the above results with previous results obtained in mammalian systems, two other P. aeruginosa strains were also examined. The PAO1-BI strain and the derived lasR mutant PAO-R1 have been used in several mammalian virulence models (Gambello et al., 1991, J. Bacteriol., 173(9), 3000-9). Surprisingly, in our cell system it appears that Dictyostelium cells grew much better on strain PAO1-BI than on our wild-type strain PT5 used in this study C(able 2), revealing that distinct PAO1 isolates can exhibit tremendous variations in virulence. In the PAO1-BI background, the lasR mutation (PAO-R1) resulted in a fully permissive strain (Table 2). Both PAO1-BI and PAO-R1 strains were previously found to exhibit nfxC phenotypes, resulting in overexpression of the MexEF-OprN efflux system (Table 3, below). This raised the possibility that the nfxC phenotype of these strains might account for their less pronounced virulence in Dictyostelium. Therefore, the nfxC mutant PT149 derived from our wild type PT5 as well as a nalC (P1625) and a nfxB (FT648) derivative, overexpressing respectively the MexAB-OprM and the MexCD-OprJ efflux pump (Table 2) were analyzed in our model system. Interestingly, the nfxC mutant PT149, which overexpresses the MexEF-OprN efflux pump, inhibited less Dictyostelium growth than the parental strain PT5 (Table 2). On the contrary, the nalC and the nfxB mutants were as inhibitory as the wild-type (Table 2). To further characterize the relationship between the nfxC phenotype and reduced virulence, the mexE gene was inactivated in the PT149 strain. This restored virulence of the strain (PT637) to the level of the isogenic wild-type bacteria C(able 2), demonstrating that it is the overexpression of the MexEF-OprN efflux pump in PT149 that accounts for its decreased virulence.

### Example 5

### In Vitro Dictyostelium Virulence Activity Correlates Positively with Mammalian Virulence

To further establish the correlation between Dictyostelium and mammalian host systems, the efflux pump mutants PT625 (nalC), PT648 (nfxB) and PT149 (nfxC) were tested in a model of acute pneumonia in rats. Bacteria were injected into rat trachea, and virulence of strains was determined by assessing mortality and time to death. In these experiments the nfxC mutant PT149 was avirulent (100% survival) while the nfxb and nalB mutants exhibited only a slightly reduced virulence (Table 4). Inactivation of mexE also restored virulence in the nfxC mutant to wild-type levels (PT637 strain). Analysis of bacterial loads in the lungs of deceased animals confirmed that for all strains studied, death was due to a severe pneumonia (Table 4). All the mutants used in this study showed growth curves which were similar to those of the wild type, suggesting that the decreased virulence of the nfxC mutant is not due to a reduced overall fitness.

### Example 6

### Reduction of Virulence of P. Aeruginosa in Vivo by Azithromycin

Azithromycin, a macrolide antibiotic, has been reported to inhibit the quorum-sensing circuitry of P. aeruginosa, in vitro. The methods of the present invention can be used to test whether macrolides inhibit the quorum-sensing circuitry of P. aeruginosa in vivo, lead to a reduction of virulence factor production, and reduce the virulence of P. aeruginosa in vivo.

A lawn of P. aeruginosa PAO1 is grown in the presence of 2 μg/ml of azithromycin overnight The bacterial lawn is then seed with different dilutions of D. discoideum DH1-10 three days latter and the appearance of D. discoideum DH1-10 colonies is monitored. Growth of colonies of D. discoideum DH1-10 are recorded and scored between 1 and 2 (same scale as FIG. 2) as compared to a score of 0 in the absence of antibiotic.

Growth of colonies of D. discoideum DH1-10 in the presence of azithromycin is expected to be significantly greater than in the absence of azithromycin, suggesting that macrolides, such as azithromycin, inhibit the quorum-sensing circuitry of P. aeruginosa in vivo, thereby leading to a reduction of virulence factor production, and, consequently, reducing the virulence of P. aeruginosa in vivo.

TABLE 1
P. aeruginosa strains used in this study

| Strain | Genotype ^{a} | Reference |
|---|---|---|
| PT5 | PAO1 wild type | Laboratory collection |
| PT462 | PT5rhlR::Tn501 | Kohler et al., 2000, J. Bacteriol., 182(21), 5990-6 |
| PT498 | PT5ΔlasR::Tc ^{b} | Kohler et al., 2000, J. Bacteriol., 182(21), 5990-6 |
| PT531 | PT5rhlR::Tn501, | This study |
| | ΔlasR::Tc | |
| PT454 | PT5ΔrhlI::Tn501 | Kohler et al., 2000, J. Bacteriol., 182(21), 5990-6 |
| PT466 | PT5ΔlasI::Tc | Kohler et al., 2000, J. Bacteriol., 182(21), 5990-6 |
| PT502 | PT5ΔrhlI::Tn501, | This study |
| | ΔlasI::Tc | |
| PT712 | PT5rhlA::Gm | Kohler et al., 2000, J. Bacteriol., 182(21), 5990-6 |
| PT648 | PT5nfxB | Kohler, 1997, Mol. Microbiol., 23(2), 345-54 |
| PT625 | PT5nalC | Kohler, 1997, Mol. Microbiol., 23(2), 345-54 |
| PT149 | PT5nfxC(previously | Kohler, 1997, Mol. Microbiol., 23(2), 345-54 |
| | called PAO-7H) | |
| PT637 | PT149, mexE::Hg | Kohler, 1997, Mol. Microbiol., 23(2), 345-54 |
| PAO1-B.I. | PAO1, nfxC | Gambello et al., 1991, J. Bacteriol., 173, 3000-9 |
| PAO-R1 | PAO1, nfxC, ΔlasR::Tc | Gambello et al., 1991, J. Bacteriol., 173, 3000-9 |
| PDO100 | ΔrhlI::Tn501 | Brint, et al., 1995, J. Bacteriol., 177(24), 7155-63 |

^{a} Except PAO1-B.I., PAO-R1 and PDO100, all the strains used in this study are isogenic to the wild-type PAO1 strain (Holloway et al., 1979 Microbiol. Rev. 43, 73-102). All strains used were tested for resistance to antibiotics, and unless specifically stated they exhibited no overexpression of antibiotic efflux systems. Mutants PT531 and 502 were generated using the transducing phage E79tv2.
^{b} Tc, tetracyline; Gm, gentamicin

TABLE 2
Growth of D. discoideum in the presence of various P. aeruginosa mutants

| | Growth substrates^{a} | | |
|---|---|---|---|
| | K. pneumoniae + | | |
| | P. aeruginosa | P. aeruginosa | P. aeruginosa^{b} |
| PAO1 | − | − | 0 |
| PT462 (rhlR) | + | − | 5 |
| PT498 (lasR) | − | − | 1 |
| PT531 (rhlR, lasR) | + | + | 8 |
| PT454 (rhlI) | + | − | 4 |
| PT466 (lasI) | − | − | 1 |
| PT502 (rhlI, lasI) | + | + | 8 |
| PT712 (rhlA) | + | − | 2 |
| PT625 (nalC) | − | − | 0 |
| PT648 (nfxB) | − | − | 1 |
| PT149 (nfxC) | + | − | 2 |
| PAO-BI (nfxC) | + | − | 4 |
| PAO-R1 | + | + | 8 |
| (lasR, nfxC) | | | |

^{a}Clonal growth of D. discoideum was tested on a mixture of K. pneumoniae and P. aeruginosa, as described in FIG. 1, or on a lawn of P. aeruginosa alone. Only robust growth was recorded as positive.
^{b}The ability of D. discoideum cells to form colonies was also tested more quantitatively on a lawn of pure P. aeruginosa as described in FIG. 2.

TABLE 3

| | MIC profiles of the strains used in this study | | | | | |
|---|---|---|---|---|---|---|
| | MIC (μg/ml)^{a} | | | | | |
| | | PT149 | PAO-BI | | PT625 | PT648 |
| Antibiotic | PAO1 | (nfxC) | (nfxC) | PDO100 | (nalB) | (nfxB) |
| Ciproflox- | 0.125 | 1 | 1 | 0.125 | 0.5 | 1 |
| acin | | | | | | |
| Chloroam- | 32 | 1064 | 1064 | 32 | 64 | 64 |
| phenicol | | | | | | |
| Aztreonam | 4 | 1 | 2 | 2 | 16 | 2 |
| Cefpirone | 2 | 0.5 | 0.5 | 2 | 4 | 8 |
| Imipenem | 1 | 4 | 4 | 1 | 1 | 1 |

^{a}MICs for strain PAO-R1 were identical to those for strain PAO-BI. All strains derived from PAO1 and not indicated here exhibited MIC profiles identical to PAO1.

TABLE 4
Virulence of P. aeruginosa efflux mutants in a rat model of acute pneumonia

| | | | | | | |
|---|---|---|---|---|---|---|
| | | | | | | Bacterial |
| | | | | | Lung | counts in |
| | Inoculum | | Mortality | Time to | weight¹ | lungs log |
| Strain | (cfu × 10⁻⁶) | n | (%) | death (Days) | (g) | cfu/g |

| PT5 (WT) | 3.0 | 22 | 72 | 1.6 ± 0.63 | 3.98 ± 0.50 | 8.30 ± 0.54 |
|---|---|---|---|---|---|---|
| PT625 (nalC) | 1.7 | 18 | 50 | 2.1 ± 1.0 | 3.84 ± 0.9 | 8.53 ± 0.38 |
| PT648 (nfxB) | 2.1 | 11 | 45 | 1.6 ± 0.89 | 4.83 ± 1.35 | 8.85 ± 0.28 |
| PT149 (nfxC) | 2.1 | 18 | 0 | NR | NR | NR |
| PT637 (NfxC, | 0.9 | 12 | 42 | 2.8 ± 1 | 4 ± 1.21 | 8.44 ± 0.48 |
| The model of acute P. aeruginosa pneumonia is based on the model by Cash and has been modified as described (Join-Lambert et al., 2001, Antimicrob. Agents Chemother., 45(2):571-6). Briefly, bacteria (10⁶ cfu) were injected in agar enmeshed beads into anesthetized male rats via the transtracheal route. Animals develop an acute bronchopneumonia, which can be fatal. Virulence of strains was determined by comparing mortality and time to death. For deceased animals, the bacterial load in | | | | | | |
| # the lungs was determined to ascertain that death of the animals was due to a severe pneumonia. | | | | | | |

¹Lung weight of uninfected rat: 1 g.
NR: not relevant.

### Equivalents

From the foregoing detailed description of the specific embodiments of the invention, it should be apparent that a unique method to identify a compound which reduces the virulence of a pathogen or toxin to a host organism or genes encoding factors that are pathogens or toxins to a host organism has been described Although particular embodiments have been disclosed herein in detail, this has been done by way of example for purposes of illustration only, and is not intended to be limiting with respect to the scope of the appended claims which follow. In particular, it is contemplated by the inventor that various substitutions, alterations, and modifications may be made to the invention without departing from the spirit and scope of the invention as defined by the claims. For instance, the choice of the particular pathogen or toxin, or combination of pathogens or toxins, or the particular unicellular test host organism or the method of exposition of the pathogen or toxin to the unicellular test host organism is believed to be a matter of routine for a person of ordinary skill in the art with knowledge of the embodiments described herein.

What is claimed is:

1. A method for assessing the virulence of a pathogen to a host organism, said method comprising:
(a) exposing a culture of unicellular test organism to the pathogen; and
(b) monitoring the growth of the test organism in the presence of the pathogen,
wherein inhibition of the growth of the test organism indicates virulence of the pathogen to the host organism.

2. The method of claim 1, wherein the test organism is an amoeba.

3. The method of claim 2, wherein the amoeba is an Entamoeba species or an Acanthamoeba species.

4. The method of claim 2, wherein the amoeba is a Dictyostelium species.

5. The method of claim 4, wherein the Dictyostelium species is D. discoideum.

6. The method of claim 5, wherein D. discoideum is D. discoideum DH1.

7. The method of claim 1, wherein the pathogen is a toxin.

8. The method of claim 1, wherein the pathogen is selected from the group consisting of:
bacteria, mycobacteria, fungi, and unicellular eukaryotic organism.

9. The method of claim 8, wherein the bacterial pathogen is an extracellular bacterial pathogen or an intracellular bacterial pathogen.

10. The method of claim 9, wherein the extracellular bacterial pathogen is Pseudomonas.

11. The method of claim 10, wherein the extracellular bacterial pathogen is P. aeruginosa.

12. The method of any one of claims 9 to 11, wherein the virulence of the bacterial pathogen is triggered by a quorum sensing pathway.

13. The method of any one of claims 1 to 12, wherein the host organism is a vertebrate.

14. The method of claim 1, wherein the unicellular test organism is exposed independently to two or more concentrations of the pathogen.

15. A method for comparing the virulence to a host organism of a first pathogen and a second pathogen, said method comprising:
(a) exposing the unicellular test organism to a first pathogen and monitoring the growth of the test organism in the presence of the first pathogen;
(b) exposing the test organism to a second pathogen and monitoring the growth of the test organism in the presence of the second pathogen; and
(c) comparing the level of growth inhibition induced by the first pathogen in step(a) with the level of growth inhibition induced by the second pathogen in step(b),
wherein the pathogen exhibiting the higher level of inhibition in step (c) has a higher virulence to the host organism.

16. A method for identifying a composition that reduces the virulence of a pathogen to a host organism, said method comprising:
(a) exposing the unicellular test organism to the pathogen in the presence of a candidate composition; and
(b) exposing the test organism to the pathogen in the absence of the candidate composition; and
(c) monitoring the growth of the test organism in the presence and in the absence of the candidate composition,
wherein a higher level of growth of the test organism in the presence of the candidate composition than observed in the absence of the candidate composition indicates that the candidate composition reduces the virulence of the pathogen to the host organism.

17. The method of claim 16, wherein the test organism is an amoeba.

18. The method of claim 17, wherein the amoeba is an Entamoeba species or an Acanthamoeba species.

19. The method of claim 17, wherein the amoeba is a Dictyostelium species.

20. The method of claim 19, wherein the Dictyostelium species is D. discoideum.

21. The method of claim 20, wherein D. discoideum is D. discoideum DH1.

22. The method of claim 16, wherein the pathogen is a toxin.

23. The method of claim 16, wherein the pathogen is selected from the group consisting of:
bacteria, mycobacteria, fungi, and unicellular eukaryotic organism.

24. The method of claim 23, wherein the bacterial pathogen is an extracellular bacterial pathogen or an intracellular bacterial pathogen.

25. The method of claim 24, wherein the extracellular bacterial pathogen is Pseudomonas.

26. The method of claim 25, wherein the extracellular bacterial pathogen is P. aeruginosa.

27. The method of any one of claims 24 to 26, wherein the virulence of the bacterial pathogen is triggered by a quorum sensing pathway.

28. The method of any one of claims 16 to 27, wherein the host organism is a vertebrate.

29. The method of claim 16, wherein the unicellular test organism is exposed independently to two or more concentrations of the pathogen.

30. The use of D. discoideum to identify a composition that reduces the virulence of a pathogen to a host organism, whereby the reduction of virulence is reproducible in a rat mortality assay.

31. A method for identifying a mutation in a virulence factor of a pathogen of a host organism that reduces the virulence of the pathogen to the unicellular host organism, said method comprising:
(a) selecting a pathogen which inhibits the growth of the unicellular test organism;
(b) generating a mutant of the pathogen;
(c) exposing a first culture of the test organism to the pathogen of step (a) and monitoring the growth of the first culture of test organism;
(d) exposing a second culture of the test organism to the mutant of step (b) and monitoring the growth of the second culture of test organism; and
(e) comparing the growth of the first culture with the growth of the second culture,
wherein a higher level of growth of the second culture of test organism than observed with first culture indicates that the mutant has a mutation in the virulence factor of the pathogen which reduces the virulence of the pathogen to the host organism.

32. The method of claim 31, wherein the test organism is an amoeba.

33. The method of claim 32, wherein the amoeba is an Entamoeba species or an Acanthamoeba species.

34. The method of claim 32, wherein the amoeba is a Dictyostelium species.

35. The method of claim 34, wherein the Dictyostelium species is D. discoideum.

36. The method of claim 35, wherein D. discoideum is D. discoideum DH1.

37. The method of claim 31, wherein the pathogen is a toxin.

38. The method of claim 31, wherein the pathogen is selected from the group consisting of:
bacteria, mycobacteria, fungi, and unicellular eukaryotic organism.

39. The method of claim 38, wherein the bacterial pathogen is an extracellular bacterial pathogen or an intracellular bacterial pathogen.

40. The method of claim 39, wherein the extracellular bacterial pathogen is Pseudomonas.

41. The method of claim 40, wherein the extracellular bacterial pathogen is P. aeruginosa.

42. The method of any one of claims 39 to 41, wherein the virulence of the bacterial pathogen is triggered by a quorum sensing pathway.

43. The method of any one of claims 31 to 42, wherein the host organism is a vertebrate.

44. The method of claim 31, wherein the unicellular test organism is exposed independently to two or more concentrations of the pathogen.
